(19) United States
(12) Patent Application Publication
(10) Pub. No.: US 2022/0324838 A1
(43) Pub. Date: Oct. 13, 2022

(54) An Improved Process Of Preparation Of Ivosidenib

(71) Applicant: Alembic Pharmaceuticals Limited, Vadodara (IN)

(72) Inventors: Vittal Tvsk, Vadodara (IN); Sanjiv Tomer, Vadodara (IN); Kamlesh Kanzariya, Vadodara (IN); Harsh Patel, Vadodara (IN)

(73) Assignee: Alembic Pharmaceuticals Limited, Vadodara (IN)

(21) Appl. No.: 17/633,696

(22) PCT Filed: Aug. 6, 2020
(86) PCT No.: PCT/IB2020/057432

§ 371 Date: Feb. 8, 2022

(30) Foreign Application Priority Data
Aug. 9, 2019 (IN) 201921032388
Feb. 14, 2020 (IN) 202021006535

(51) Int. Cl. C07D 401/14; A61K 31/444

(52) U.S. Cl. CPC C07D 401/14; A61K 31/444

(57) ABSTRACT

The present invention relates to an improved process of preparation of Ivosidenib a compound of Formula (I). More particularly, present invention provides process of preparation of intermediates. Also provide process of preparation of amorphous form of Ivosidenib. Further, present invention provides process of preparation of chirally pure Ivosidenib a compound of Formula (I).

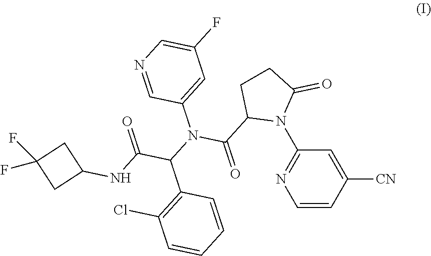 [SMILES: N#Cc1ccnc(N2C(=O)CCC2C(=O)N(c2cncc(F)c2)C(C(=O)NC2CC(F)(F)C2)c2ccccc2Cl)c1]

### RELATED APPLICATION

This application claims the benefit of the earlier filing date of Indian Provisional Patent Application No. 201921032388 filed on Aug. 9, 2019 and Indian Provisional Patent Application No. 202021006535 filed on Feb. 14, 2020.

### FIELD OF THE INVENTION

The present invention relates to an improved process of preparation of Ivosidenib of formula (I).

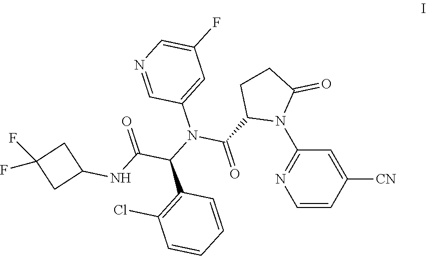

### BACKGROUND OF THE INVENTION

Ivosidenib, has a chemical name (2S)—N-{(1S)-1-(2-chlorophenyl)-2-[(3,3-difluoro cyclobutyl)-amino]-2-oxoethyl}-1-(4-cyanopyridin-2-yl)-N-(5-fluoropyridin-3-yl)-5-oxo pyrrolidine-2-carboxamide. Ivosidenib is represented by the following chemical structure according to Formula (I).

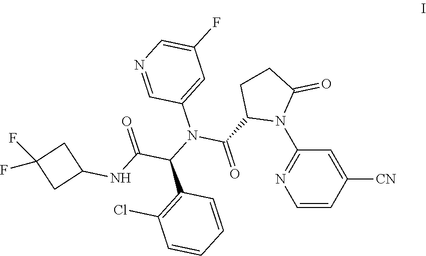

Ivosidenib is an orally-available, small molecule inhibitor that targets the mutant isocitrate dehydrogenase 1 (IDH1) enzyme. Ivosidenib was shown to inhibit selected IDH1 R132 mutants at much lower concentrations than wild-type IDH1 in vitro. Inhibition of the mutant IDH1 enzyme by Ivosidenib led to decreased 2 HG levels and induced myeloid differentiation in vitro and in vivo in mouse xenograft models of IDH1-mutated AML. In blood samples from patients with AML with mutated IDH1, Ivosidenib decreased 2-HG levels ex-vivo, reduced blast counts, and increased percentages of mature myeloid cells.

U.S. Pat. No. 9,474,779 discloses the preparation of Ivosidenib by the process as depicted in Scheme I:

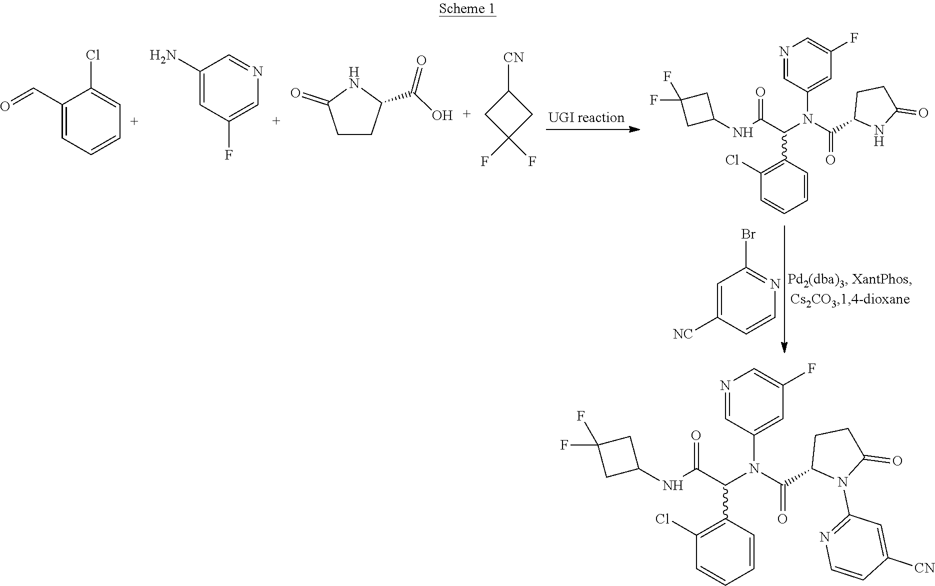

Polymorphic forms of Ivosidenib are disclosed in WO2015138839 and WO2020010058. Further, WO2019104318 discloses polymorphs as well as process for preparation of Ivosidenib.

Considering the importance of Ivosidenib in the pharmaceutical field, there is a need remains for an improved and commercially viable process of preparing chiraly pure Ivosidenib.

### SUMMARY OF THE INVENTION

An aspect is to provide an improved process of preparation of Ivosidenib, comprising the steps of:
a) converting compound of Formula (III) to compound of Formula (IV) in presence of phosgene derivative;

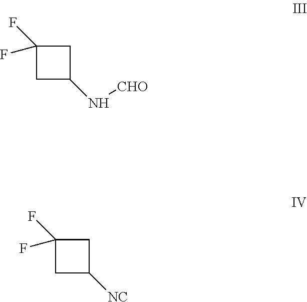

b) converting the compound of Formula (IV) into compound of Formula (IX) under reactions conditions similar to UGI reaction;

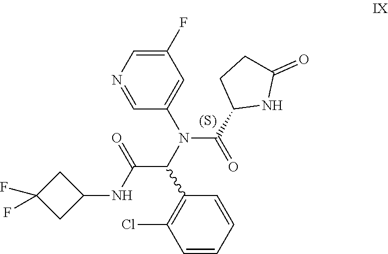

c) reacting compound of Formula (IX) with compound of Formula (X) to obtain racemic Ivosidenib a compound of Formula (XI) in the presence of solvent; wherein X is a leaving group; and

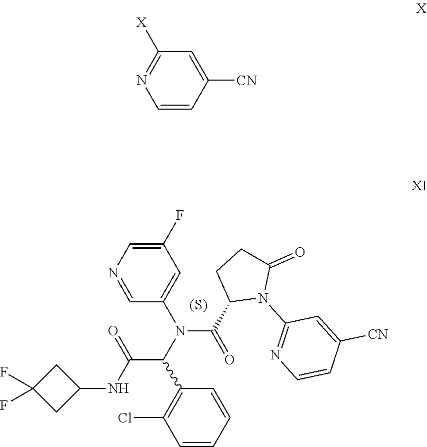

d) converting compound of Formula (XI) in to Ivosidenib of Formula (I).

Another aspect is to provide an improved process of preparation of Ivosidenib, comprising the reacting compound of Formula (IX) with compound of Formula (X) to obtain racemic Ivosidenib a compound of Formula (XI) in the presence of solvent containing water, wherein X is a leaving group; and

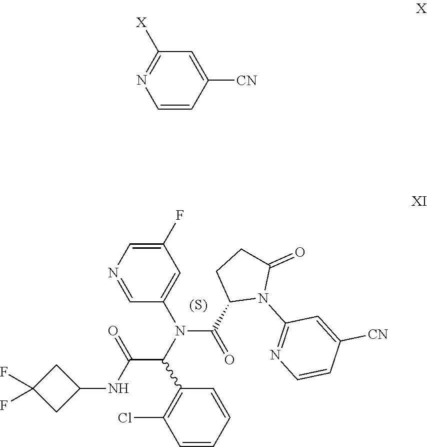

converting compound of Formula (XI) in to Ivosidenib of Formula (I).

Yet another aspect is to provide an improved process of preparation of Ivosidenib, comprising converting compound of Formula (III) to compound of Formula (IV) in presence of phosgene derivative; and

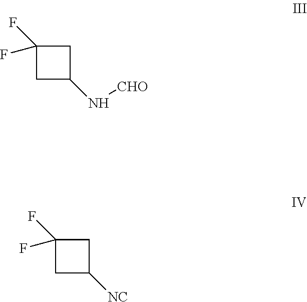

converting compound of Formula (IV) in to Ivosidenib of Formula (I).

One more aspect of the invention is to provide a resolution method of compound of formula (XI) as described herein.

### BRIEF DESCRIPTION OF THE DRAWINGS

FIG. 1 shows the X-ray powder diffractogram (“PXRD”) pattern of Compound of Formula (XII).

FIG. 2 shows the differential scanning calorimetry (“DSC”) pattern of Compound of Formula (XII).

### DETAILED DESCRIPTION OF THE INVENTION

In one embodiment of the present invention is to provide a process of preparation of (3,3-difluorocyclobutyl) isonitrile according to the compound of formula (IV), comprising the step of:

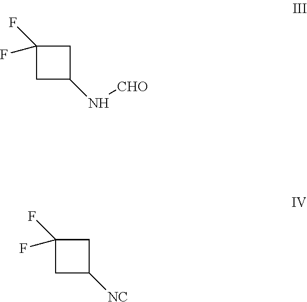

reacting (3,3-difluorocyclobutyl) formamide according to the compound of Formula (III) with phosgene derivative.

In another embodiment of the present invention, conversion of compound of Formula (III) to compound of Formula (IV) is carried out in the presence of suitable phosgene derivative such as phosgene, diphosgene or triphosgene. Phosgene and the derivative thereof may be in solid, liquid or gaseous form. The reaction may be carried out in presence of organic base such as triethyl amine, diethyl amine, diethyl isopropylamine and the like. The amount of organic base used for the reaction is about 2.5 to 3.0 mole equivalents with respect to 2-chloro benzaldehyde. The reaction may be carried out in the presence of an inert organic solvent including but not limited to solvent such as dichloromethane (DCM), chloroform, carbon tetrachloride (CCl₄), 1,2-dichloroethane, trichloroethylene, tetrahydrofuran (THF), toluene, acetonitrile, tetrachloroethylene, and mixtures thereof. The reaction may be carried out at a temperature of about −20° C. to about boiling point of the solvent used. Specifically, the reaction step (a) may be carried out at a temperature of about 25° C. to 30° C. The term inert organic solvent as described herein include any solvent that does not affect the course of the reaction.

In one embodiment, compound of Formula (IV) may be isolated and purified if required from the reaction mixture by any known technique in the art or the compound can be subjected to next reaction without isolation and/or purification. Specifically, compound can be subjected to next reaction without isolation, or purification or any work up procedure.

In another embodiment the compound of Formula (IV) is converted to compound of Formula (IX) by multicomponent reactions such as UGI reaction condition that are known in the art. For example, the typical UGI reaction involves, but not limited to, either three component or four component as explained below.

Three component UGI reaction comprises reaction of compound of Formula (IV), (V) and (VIII).

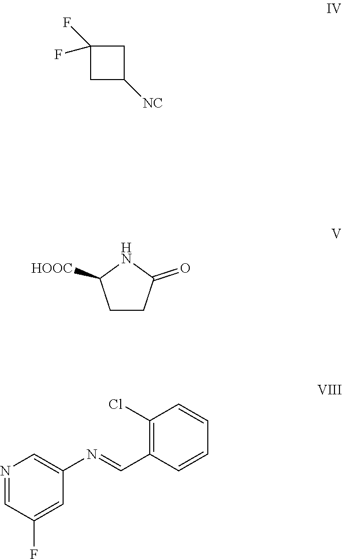

Four component UGI reaction comprises reaction of compound of Formula (IV), (V), (VI) and (VII);

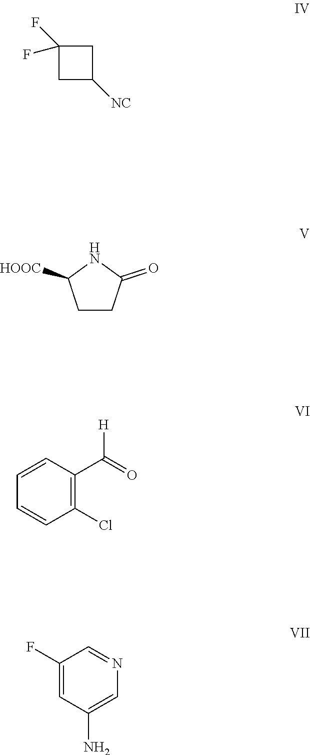

In one embodiment, compound of Formula (VIII) can be formed by reacting compound of Formula (VI) with compound of Formula (VII) in presence of suitable solvent under dehydrating conditions which includes use of molecular sieves and optionally in the presence of lewis acid. The conditions of UGI reaction are known in the art.

In one more embodiment of the present invention, reacting a compound of Formula (IX) with compound of Formula (X), wherein X is a leaving group such as chloro, bromo, iodo, tosyl and the like; preferably a halo group, to obtain compound of Formula (XI) in presence of tris(dibenzylideneacetone)dipalladium and xantphos with base such as potassium carbonate or cesium carbonate in inert solvent that includes but not limited to toluene, heptane, benzene, dioxane, chlorobenzene, anisole. The reaction is conveniently conducted in the presence of small amount of water; the water increases the solubility of base so that the reaction proceeds smoothly. Small amount of water involves 0.5 volume to 1.5 volume of water. The reaction can be carried out either in open system or closed system; wherein closed system can be autoclave or sealed tube. Optionally, the reaction can be carried out in presence of catalytic amount of KI or phase transfer catalyst.

A compound of Formula (XI), which is represented as a racemic Ivosidenib, is a racemic mixture of undesired isomer of Formula (XII) ((2S)—N-((1R)-1-(2-chlorophenyl)-2-((3, 3-difluoro cyclobutyl) amino)-2-oxoethyl)-1-(4-cyanopyridine-2yl)-N-(5-fluoropyridin-3-yl)-5-oxopyrrolidine-2-carboxamide) and desired isomer of Formula (I) ((2S)—N-((1S)-1-(2-chlorophenyl)-2-((3, 3-difluoro cyclobutyl) amino)-2-oxoethyl)-1-(4-cyanopyridine-2yl)-N-(5-fluoropyridin-3-yl)-5-oxopyrrolidine-2-carboxamide). The resolution of Formula (XI) is carried out selective crystallization method as described herein. The racemic compound of Formula (XI) can be separated by selective crystallization to obtain either desired isomer (I) OR undesired isomer (XII) as a solid material whereas mother liquor (or filtrate) containing other of desired/undesired isomer can be enriched or epimerized further to obtain desired/undesired isomer in presence of suitable base and optionally in presence of solvent, which can be recycled and subjected to separation. Suitable base used is such as DBU (1,8-Diazabicyclo[5.4.0]undec-7-ene), DABCO ((1,4-diazabicyclo[2.2. 2]octane)), DIPEA (N,N-Diisopropylethylamine), N-methyl morpholine, piperazine, piperidine, cesium carbonate, Tripotassium phosphate in suitable solvent is such as THF, DCM, ethyl acetate, toluene, methanol, acetonitrile and acetone like polar and non-polar solvents or mixtures thereof. The epimerization can also be done in neat DBU without solvent. The epimerization (or enriching of isomer) can also be done under acidic conditions. Thus the process of epimerization involves enriching the undesired diastereomer to desired diastereomer or vice-versa.

As used herein, a compound is “enriched” in a particular diastereomer when that diastereomer is present in excess over any other diastereomer present in the compound. Where a mixture is enriched in one diastereoisomeric or diastereomeric pair relative to the other diastereoisomeric or diastereomeric pair, the mixture is enriched in the depicted or referenced diastereoisomeric or diastereomeric pair relative to the other diastereoisomeric or diastereomeric pair of the compound, e.g., in a molar excess of at least 55%, 75%, 90%, 95%, 99% or 99.5%. The amount of enrichment can be determined using conventional analytical methods routinely used by those of ordinary skill in the art, including but not limited to, NMR spectroscopy in the presence of chiral shift reagents, gas chromatographic analysis using chiral columns, and high pressure liquid chromatographic analysis using chiral columns.

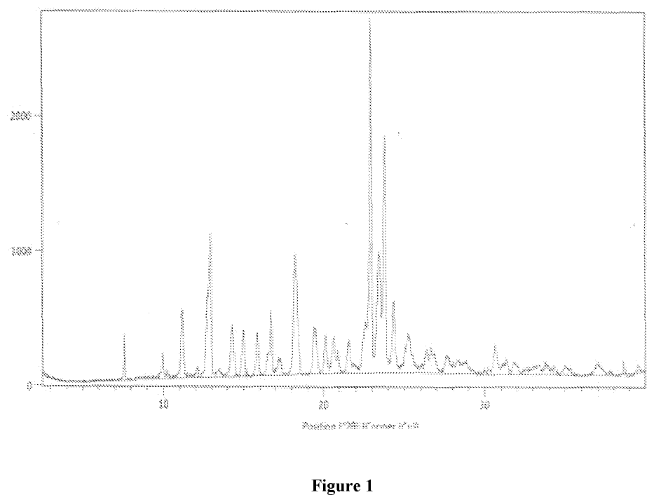

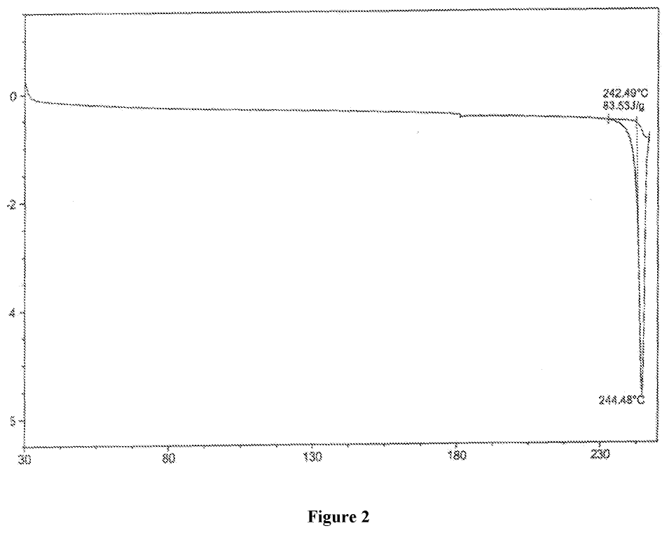

One more embodiment of the invention relates to selective crystallization of undesired isomer of Formula (XII) by treating the racemic mixture of Formula (XI) with solvent such as toluene, ethanol, isopropanol, MTBE (methyl tert-butyl ether), anisole, chlorobenze, ethyl acetate or mixtures thereof; preferably using toluene. The undesired isomer isolated is epimerized under suitable condition such by treatment with base and solvent as described above; preferably by treating with DBU in ethyl acetate or THF solvent. A solid state form of Formula (XII) obtained here can be characterized by an XRPD pattern as depicted in FIG. 1. The obtained crystalline form may be further characterized by the XRPD pattern having characteristics peaks at 7.5, 11.1, 12.9, 14.2, 14.9, 16.7, 18.1, 19.3, 22.0, 23.4 and 23.7 degree 2theta and DSC as depicted in FIG. 2.

Accordingly the present invention provides a process for the resolution of racemic Ivosidenib formula (XI) which comprises
a) obtaining a solution of racemic compound of formula (XI) in a solvent;
b) selectively crystallizing compound of formula (XII) (undesired isomer);
c) separating the compound of formula (XII) by filtration and collecting the mother liquor (filtrate);
d) optionally epimerizing the compound of (XII) and repeating step (a) to (c);
e) isolating the Ivosidenib of Formula (I) from a mother liquor of step (c) or (d).

In another embodiment of the invention, obtaining a solution racemic compound of formula (XI) in a solvent includes obtaining the solution directly by a reaction medium or by dissolving the racemic compound of formula (XI) in a solvent selected from toluene, ethanol, isopropanol, MTBE, anisole, chlorobenze, ethyl acetate or mixtures thereof; preferably toluene. The crystallization of crystallizing compound of formula (XII) (undesired isomer) is effected by cooling the step (a) solution or by concentrating step (a) solution to minimum level as to induce crystallization.

The crystallizing compound of formula (XII) (undesired isomer) is epimerized by using described the condition above. The term epimerization or epimerized indicates enhancement of other isomer. The epimerized compound is converted Ivosidenib of formula (I) by following the step (a) to (c). Repeating the steps one or more times found to be helpful in increasing the yield of the compound of formula (I). Ivosidenib of Formula (I) is isolated from mother liquor by conventional methods such as concentrating followed by crystallization by cooling and/or antisolvent addition. Crystallization can be induced with seed.

In one more embodiment the desired isomer can be selectively crystallize from a suitable solvent like ethyl acetate or mixture of ethyl acetate and heptane.

Still another embodiment, the invention relates to selective crystallization of compound of formula (XII) comprises isolation of the compound of formula (XII) from a solution of compound of formula (XI) in a solvent. The crystallization of crystallizing compound of formula (XII) (undesired isomer) is effected by cooling the step (a) solution or by concentrating step (a) solution to minimum level as to induce crystallization; preferably by reducing the solution volume by concentration.

Alternatively, the racemic compound of Formula (XI) can also be effectively resolved by resolution of its diastereomeric salts obtained using optically active acids or bases and liberation of optically active acidic or basic compounds. For example, a racemic mixture a compound of Formula (XI) treated with optically active resolving agents such as tartaric acid, mandelic acid, dibenzoyl-tartaric acids, di-O, O′-p-toluoyltartaric acid, mandelic acid and camphor sulphonic acid to obtain salt of desired isomer and converting it to Ivosidenib.

Further, efficient resolution process for preparation of Ivosidenib in desired isomeric form provides reacting a racemic mixture a compound of Formula (XI) with organic acid such as but not limited to succinic acid, oxalic acid, adipic acid to obtain salt of desired isomer and converting it to Ivosidenib.

In one of the embodiment, process of preparation of an amorphous form of Ivosidenib, comprising the steps of:
a. providing a solution of Ivosidenib with suitable acid optionally in presence of solvent; and
b. isolating amorphous form of Ivosidenib.

In an embodiment of the invention, providing a solution of Ivosidenib in a suitable acids achieved by dissolving Ivosidenib in a suitable acid, or such a solution may be obtained in the course of its synthesis as a residue which is dissolved in suitable acid or such a solution may be obtained in the course of its synthesis to which suitable acid is added. Providing a solution of Ivosidenib optionally involves use of other suitable solvents capable of dissolving Ivosidenib.

In still another embodiment of the present invention, the suitable acid is selected from fumaric acid, formic acid, maleic acid, succinic acid, adipic acid, propionic acid, malonic acid, citric acid, acetic acid, and the like or mixtures thereof and the suitable solvent include any solvent that can dissolve the Ivosidenib.

In another embodiment of the present invention the isolation of Ivosidenib, preferably in the form of amorphous, is effected by contacting the solution with a suitable base is selected from the group consisting of potassium acetate, sodium hydroxide, potassium hydroxide, potassium phosphate, sodium phosphate, sodium bicarbonate, ammonia, ammonium hydroxide, sodium ammonia, sodium and carbonate. The base may be added either in the form of solid or in the form of solution.

In another embodiment of the present invention the isolation of Ivosidenib, preferably in the form of amorphous, by adding suitable antisolvent like water, preferably cold water. The temperature during addition is maintained at about 0-5 deg C.

Also, provided herein is a process for preparing amorphous form of Ivosidenib, by dissolving the Ivosidenib in a suitable solvent or mixture thereof and removal of solvent by any known method in the art like concentration or spary drying; and isolationg the amorphous form.

The isolation of amorphous form of Ivosidenib can be carried out by employing any of the techniques known in the art. Techniques for the isolation of amorphous form of Ivosidenib include, but not limited to: decantation, filtration by gravity or suction, evaporation of the solvent, concentrating the solution centrifugation, and the like, and optionally washing with a solvent.

In some embodiments the isolation of amorphous may be effected by removal of the solvent, the removal of solvent comprises one or more of distillation, distillation under vacuum, spray drying, agitated thin film drying (“ATFD”), and freeze drying (lyophilization).

The amorphous form is isolated by slurring with non-polar solvent to obtain amorphous Ivosidenib. The solvent used for dissolution includes but not limited to and selected from acetone, MTBE, ethyl formate and the non-polar solvent is selected from heptane, cyclohexane, hexanes and the like.

In one embodiment, the present invention provides a process for the preparation of compound of Formula (XI) as represented schematically in scheme 2 as shown below.

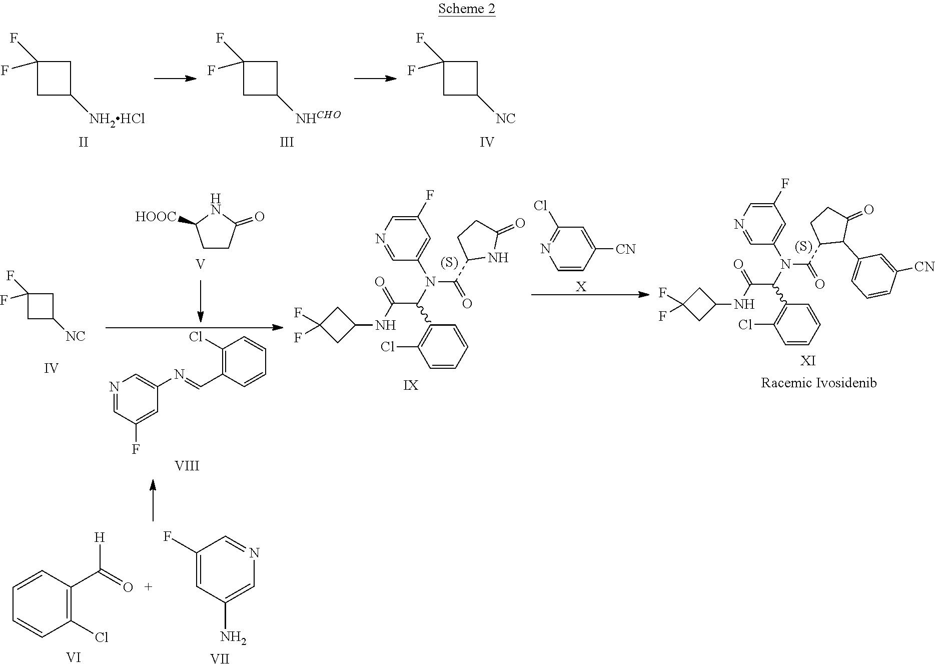

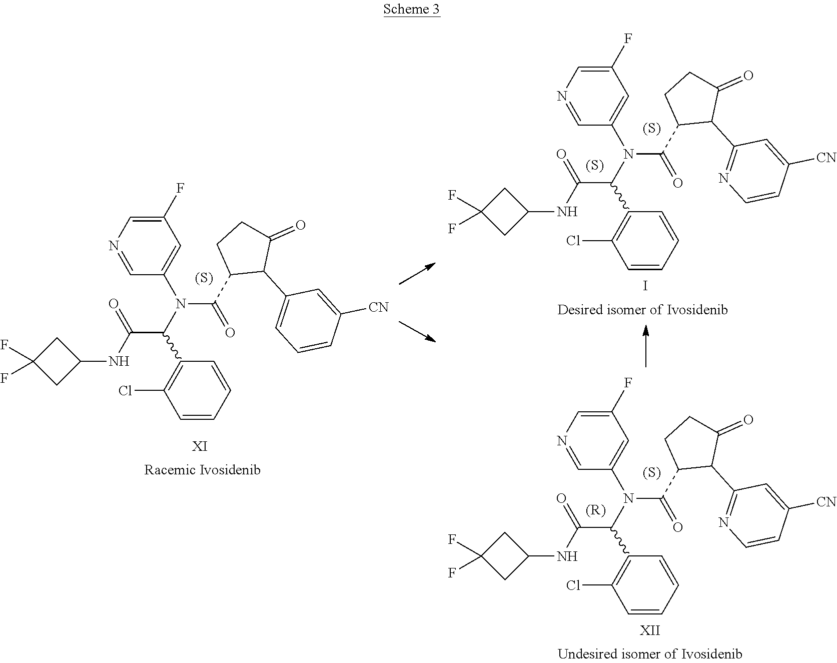

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of the person skilled in the art.

Wherever applicable in the example of the present invention, the reaction solution may optionally be treated with carbon, flux-calcined diatomaceous earth (Hyflow) or any other suitable material like N-acetyl-L-cysteine, SilaMetS thiol to remove metallic impurity, color, insoluble materials, improve clarity of the solution, and/or remove impurities adsorbable on such material. Optionally, the solution obtained above may be filtered to remove any insoluble particles. The insoluble particles may be removed suitably by filtration, centrifugation, decantation, or any other suitable techniques under pressure or under reduced pressure. The solution may be filtered by passing through paper, glass fiber, cloth or other membrane material, or a bed of a clarifying agent such as Celite® or Hyflow. Depending upon the equipment used and the concentration and temperature of the solution, the filtration apparatus may need to be preheated to avoid premature crystallization.

The isolated compound according to the present invention may be recovered by methods including decantation, centrifugation, evaporation, gravity filtration, suction filtration, or any other technique for the recovery of solids under pressure or under reduced pressure. The recovered solid may optionally be dried. Drying may be carried out in a tray dryer, vacuum oven, air oven, cone vacuum dryer, rotary vacuum dryer, fluidized bed dryer, spin flash dryer, flash dryer, or the like. The drying may be carried out at temperatures less than about 100° C., less than about 80° C., less than about 60° C., less than about 50° C., less than about 30° C., or any other suitable temperatures, at atmospheric pressure or under a reduced pressure, as long as the compound is not degraded in quality. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller and hammer milling, and jet milling

### EXAMPLES

### Example-1: Preparation of (2S)—N-(1-(2-chlorophenyl)-2((3,3-difluorocyclobutyl) amino)-2-oxoethyl)-N-(5-fluoropyridin-3-yl)-5-oxopyrrolidine-2-carboxamide (IX)

In trifluoroethanol (500 ml), 2-chloro-benzaldehyde (82.6 g) and 5-fluoropyridin-3-amine (66.1 g) was added at 30±5° C. and stirred for 10 minutes. Reaction mixture was distilled till approximately 450 ml volume remains in reaction mass. Further distillation continued with simultaneously addition of fresh trifluoroethanol. Obtained reaction mixture was cooled to 43±3° C. under nitrogen, followed by addition of trifluoroethanol (1000 ml) and L-pyroglutamic acid (115.7 g) at 43±3° C., stirred and cooled to 30±5° C. (solution A). In another reaction vessel, to a mixture of dichloromethane (175 ml) and triethylamine (170 g); N-(3,3-difluorocyclobutyl)formamide (100 g) was added and stirred to get clear solution and reaction mixture was cooled to −7±10° C. followed by slow addition of triphosgene solution (80.1 g in 150 ml MDC) at −10 to −15° C. The reaction mass was stirred at 7±3° C. till completion of reaction to obtain reaction mass contains compound of formula (IV). Earlier prepared clear solution (solution A) was added in the above reaction mass containing compound of formula (IV) at 20±15° C. and stirred at 30±5° C. till completion of reaction. After completion of reaction, water was added to obtained reaction mass and both layers were separated and organic layer was distilled out. The obtained residue was dissolved in acetonitrile and stirred at 75±5° C., cooled at 5±5° C. and then filtered. Obtained wet solid was slurred in toluene at 28±5° C. and then filtered to give compound of formula (IX) (170-205 g).

### Example-2: Preparation of (2S)—N-((1S)-1-(2-chlorophenyl)-2-((3, 3-difluoro cyclobutyl)amino)-2-oxoethyl)-1-(4-cyanopyridine-2yl)-N-(5-fluoropyridin-3-yl)-5-oxo pyrrolidine-2-carboxamide (I)

To the stirred solution of tris(dibenzylideneacetone)dipalladium (0) (1.2 g) and xantphos (3.0 g) in toluene (800 ml), compound of formula (IX) (100 g) and 2-chloro-4-cyano pyridine (27.5 g) were charged followed by addition of solution of potassium carbonate (28.0 g) in DM water (100 ml) under argon. The reaction mass was stirred to 87±5° C. After completion of reaction, the reaction mixture was cooled to 60±5° C., followed by addition of activated carbon and water (350 ml), stirred and filtered. Layers were separated and in organic layer N-acetyl-L-cysteine was added. Reaction mixture was stirred at 60±5° C. and filtered. Sodium bicarbonate solution was charged in obtained filtrate, stirred and organic layer was separated. Water (500 ml) was added and the reaction mass was stirred. Toluene layer was separated and distilled up to 2-2.5 volume. The obtained reaction mass was cooled to 30±5° C. Solid was filtered (undesired isomer compound of formula (XII)) and filtrate (filtrate A) was kept aside for further use.

Obtained solid was dissolved in ethyl acetate (200 ml) and DBU (5 g) at 45±5° C. and stirred for 20 hours. Reaction mass was stirred with water and organic layer was separated and distilled under vacuum at 70° C. to 2.5-3 volume. Toluene (60 ml) was added to the obtained reaction mass, distilled out atmospherically up to 2-3 volume, cooled to 30±5° C., stirred and solid filtered to obtain filtrate (filtrate B). Filtrate A was combined with Filtrate B and treated with activated carbon and filtered. The obtained filtrate was distilled, to residue ethyl acetate (100 ml), heptane (200 ml) were added at 60° C. To the reaction mass seed of compound of formula (I) was added at 50° C. and stirred at 25±5° C. The obtained reaction mass was cooled to 5±5° C. and filtered the solid. The obtained solid was again dissolved in ethyl acetate at 30±5° C., reaction mass was treated with SilaMetS thiol and filtered. To the obtained reaction mass heptane was added at 60±5° C. and seed of compound of formula (I) at 25° C. was added and stirred at 25±5° C. The reaction mass was cooled, stirred, filtered and wet cake was dried under vacuum to obtain compound of formula (I) (50-70 g).

### Example-3: Preparation of (2S)—N-((1S)-1-(2-chlorophenyl)-2-((3, 3-difluoro cyclobutyl)amino)-2-oxoethyl)-1-(4-cyanopyridine-2yl)-N-(5-fluoropyridin-3-yl)-5-oxo pyrrolidine-2-carboxamide (I)

A compound of formula (XI) (racemic Ivosidenib) (100 g) was stirred with toluene (200 ml) at 70° C. for 1.0 hours then cooled to 30±5° C. Solid was filtered (undesired isomer compound of formula (XII)) and filtrate (filtrate A) was kept aside for further use. The solid was dried at 50° C. (Yield—25-40 g; purity 98% and chiral purity 99.9%).

The obtained solid was dissolved in ethyl acetate (200 ml) and DBU (5 g) at 45±5° C. and stirred for 20 hours. Reaction mass was stirred with water and organic layer was separated and distilled under vacuum at 70° C. to 2.5-3 volume. Toluene (60 ml) was added to the obtained reaction mass, distilled out atmospherically up to 2-3 volume, cooled to 30±5° C., stirred and solid filtered to obtain filtrate (filtrate B). Filtrate A was combined with Filtrate B and treated with activated carbon and filtered. The obtained filtrate was distilled followed by addition of ethyl acetate (100 ml), Heptane (200 ml) at 60° C., seed of compound of formula (I) at 50° C. and stirred at 25±5° C. The obtained reaction mass was cooled to 5±5° C. and filtered the solid. The obtained solid was again dissolved in ethyl acetate at 30±5° C., reaction mass was treated with SilaMetS thiol and filtered. To the obtained reaction mass heptane was added at 60±5° C. and seed of compound of formula (I) at 25° C. were added and stirred at 25±5° C. The reaction mass was cooled, stirred, filtered and wet cake was dried under vacuum to obtain compound of formula (I) (50-70 g).

### Example-4: Preparation of (2S)—N-((1S)-1-(2-chlorophenyl)-2-((3, 3-difluoro cyclobutyl)amino)-2-oxoethyl)-1-(4-cyanopyridine-2yl)-N-(5-fluoropyridin-3-yl)-5-oxo pyrrolidine-2-carboxamide (I)

To stirred solution of tris(dibenzylideneacetone)dipalladium (0) (0.95 g) and xantphos (1.2 g) in toluene (1800 ml), compound of formula (IX) (100 g) and 2-chloro-4-cyano pyridine (27.42 g) were charged followed by addition of potassium carbonate (27.35 g) under argon. The reaction mass was heated to 95±5° C. Water (3.37 g) was added and reaction mass was stirred at 95±5° C. After completion of reaction, the reaction mixture was cooled to 30±5° C. Reaction mass was cooled and water (1000 ml) was added. Layers were separated and in organic layer N-acetyl-L-cysteine (8 g) was added. Reaction mixture was stirred at 50-60° C. Water (500 ml) was added and the reaction mass was stirred. Organic layer was separated and distilled. Toluene (200 ml) was charged to residue at 60±5° C. and stirred then was cooled to 10±5° C. and stirred. The reaction mass was filtered to obtain solid (undesired isomer compound of formula (XII)).

The obtained filtrate was distilled (having desired isomer about 85% and undesired isomer about 15%) (residue A).

An undesired isomer compound of formula (XII) (diasteriomeric purity of 99.9%) was dissolved in THF (1000 ml) and DBU (12 g) at 45±5° C. and stirred for 14 hours. The reaction mass was concentrated and residue was dissolved in ethyl acetate (600 ml) at 50±5° C. The reaction mass was stirred with water (250 ml) and organic layer was separated and concentrated under vacuum at 60° C. to obtain residue (residue B). Residue A was combined with Residue B and both were dissolved in ethyl acetate (200 ml) at 60° C. Heptane (200 ml) was added and stirred at 60±5° C. The obtained reaction mass was cooled to 10±5° C. and filtered the solid. Obtained solid was again dissolved in ethyl acetate (160 ml) at 30±5° C. followed by addition of heptane (56 ml) at 60±5° C. and stirred at 30±5° C. The reaction mass was filtered and wet cake was dried under vacuum to obtain compound of formula (I) (52 g) (Diasteriomeric purity—99.94%).

### Example-5: Preparation of (S)—N—((S)-1-(2-chlorophenyl)-2-((3, 3-difluoro cyclobutyl) amino)-2-oxoethyl)-1-(4-cyanopyridine-2yl)-N-(5-fluoropyridin-3-yl)-5-oxopyrrolidine-2-carboxamide (I)

To stirred solution of tris (dibenzylideneacetone)-dipalladium (0) (76 mg) and BINAP (104 mg) in toluene (160 ml) Compound of formula (IX) (8.00 gm) and compound of formula (X) (3.2 g) were charged followed by addition of anhydrous potassium carbonate (2.8 g) under argon. The reaction mass was heated to reflux and maintained at the same temperature till completion of reaction. After completion of reaction, the reaction mixture was cooled to 50-60° C. The reaction mass in toluene layer was washed with water followed by addition of N-acetyl-L-cysteine (0.64 gm). Reaction mixture was stirred at 50-60° C. and organic layer was distilled partially under reduced pressure. The reaction mass was cooled to 28-30° C. and stirred. The solid viz undesired isomer was filtered (purity: 98%, diastereomeric purity: 99.9%; XRD; FIG. 01, DSC FIG. 02). The obtained filtrate was distilled and the residue (having desired isomer about 85% and undesired isomer about 15%) was purified by column chromatography to give desired isomer of Ivosidenib (3.5 g, diastereomeric purity about 99.9%).

### Example-6: Preparation of (S)—N—((S)-1-(2-chlorophenyl)-2-((3, 3-difluoro cyclobutyl) amino)-2-oxoethyl)-1-(4-cyanopyridine-2yl)-N-(5-fluoropyridin-3-yl)-5-oxopyrrolidine-2-carboxamide (I)

To stirred solution of tris (dibenzylideneacetone)-dipalladium (0) (76 mg) and BINAP (104 mg) in toluene (160 ml) Compound of formula (IX) (8.00 gm) and compound of formula (X) (3.2 g) were charged followed by addition of anhydrous potassium carbonate (2.8 g) under argon. The reaction mass was heated to reflux and maintained at the same temperature till completion of reaction. After completion of reaction, the mixture was cooled to 50-60° C. The reaction mass in toluene layer was washed with water followed by addition of N-acetyl-L-cysteine (0.64 gm). The reaction mixture was stirred at 50-60° C. and organic layer was distilled under reduced pressure to get residue. To the residue was added THF (80 ml) and DBU (2.5 ml) and stirred at ambient temperature for 18-24 hrs. THF was distilled and was purified by column chromatography to give desired isomer of Ivosidenib (6.0 g, diastereomeric purity about 99.9%).

### Example-7: Preparation of (S)—N-(1-(2-chlorophenyl)-2-((3,3-difluorocyclobutyl) amino)-2-oxoethyl)-1-(4-cyanopyridin-2-yl)-N-(5-fluoropyridin-3-yl)-5-oxopyrrolidine-2-carboxamide (XI)

An undesired isomer compound of compound of formula (XII) (10 g, disteriomeric purity of 99.9%) was dissolved in THF (100 ml) and DBU (3.5 g) at 25-45° C. for 48-72 hrs. The reaction mass was concentrated and residue was dissolved in ethyl acetate (300 ml) at 25-30° C. The reaction mass was washed with water (250 ml) and organic layer was separated and concentrated under vacuum at 50° C. to obtain compound of formula (XI) (10.0 g; diasteriomeric purity of compound-I is 65-100%).

### Example-8: Preparation of Benzyl Alcohol Solvate of Ivosidenib

Ivosidenib (1.0 g) was dissolved in dichloromethane (10 ml) and benzyl alcohol (1 ml) at 25-30° C. The reaction mass was concentrated and residue was stirred with cyclohexane (20 ml) at 25-30° C., filtered the solid and dried at 25-30° C. for 8 hours (0.86 g).

### Example-9: Preparation of Amorphous Form of Ivosidenib

Ivosidenib (100 g) was dissolved in acetone (800 ml) at 30±5° C. The reaction mass was concentrated then followed by slurred with cyclohexane (300 ml). The solid was filtered and washed with cyclohexane (100 ml). The solid was dried at 60°±5 C for 8 hours (85-90 g).

### Example-10: Preparation of Amorphous Form of Ivosidenib

Ivosidenib (100 g) was dissolved in acetone (1600 ml) at 30±5° C. The reaction mass was spray dried. The solid was dried at 60°±5 C for 8 hours (85-90 g).

### Example-11: Preparation of Amorphous Form of Ivosidenib

Ivosidenib (100 g) was dissolved in formic acid (170 ml) at 30±5° C. The reaction mass was treated with activated carbon and stirred. The obtained reaction mass was filtered through hyflo bed and wash with formic acid (30 ml). The obtained reaction mass was quenched with cold water (1250 g) at −5 to 5° C., stirred and filtered. The obtained solid was slurred in water and filtered. (85-90 g).

### Example-12: Preparation of Amorphous Form of Ivosidenib

Ivosidenib (100 g) was dissolved in formic acid (180 ml) at 30±5° C. The reaction mass was filtered through hyflo bed and wash with acetone (20 ml). To the obtained reaction mass ammonia solution (1250 ml) was added, stirred and filtered. The obtained solid was slurred in water and filtered. The solid was dried at 25°±5 C (85-90 g).

### Example-13: Preparation of Amorphous Form of Ivosidenib

Ivosidenib (1 g) was dissolved in ethyl formate (1.6 ml) at 52±5° C. To the obtained reaction mass n-hexane (3 ml) was added and cooled to 20° C. and filtered. The solid was dried under vacuum (800 mg).

### Example-14: Preparation of Amorphous Form of Ivosidenib

Ivosidenib (1.2 g) was dissolved in MTBE (1.2 ml) at 52±5° C. To the obtained reaction mass n-hexane (3.6 ml) was added and cooled to 20° C. and filtered. The solid was dried under vacuum (850 mg).

### Example-15: Preparation of Amorphous Form of Ivosidenib

Ivosidenib (1.2 g) was dissolved in MTBE (1.3 ml) at 52±5° C. To the obtained reaction mass cyclohexane (3.6 ml) was added and cooled to 20° C. and filtered. The solid was dried under vacuum (800 mg).

### Example-16: Preparation of N-(3,3-difluorocyclobutyl) formamide (III)

To a mixture of ethylformate (700 ml) and a compound of formula (II) (100 g) in autoclave, triethylamine (195 g) was added and reaction mass was maintained at 85±5° C. After completion of reaction, reaction mass was cooled to 30±5° C. the solid was filtered and washed with ethyl acetate. The obtained filtrate was treated with sodium bicarbonate solution and layers were separated. Water was added to the obtained organic layer; organic layer was separated and concentrated. Cyclohexane (or similar non polar solvent) was added in the reaction mass and was stripped out. The obtain reaction mass was stirred with cyclohexane and filtered the solid, to obtained compound of formula (III) (90 g).

1.-25. (canceled)

26. A process for preparation of amorphous form of Ivosidenib comprising the steps of:
(a) providing a solution of ivosidenib with suitable acid, optionally in presence of solvent; and
(b) contacting the step (a) solution with base or with an anti-solvent at a temperature in the range of 0-5 deg C.

27. The process as claimed in claim 26, wherein the suitable acid is selected from fumaric acid, formic acid, maleic acid, succinic acid, adipic acid, propionic acid, malonic acid, citric acid, acetic acid or mixtures thereof.

28. The process as claimed in claim 27, wherein the acid is formic acid.

29. The process as claimed in claim 26, wherein the base is selected from potassium acetate, sodium hydroxide, potassium hydroxide, potassium phosphate, sodium phosphate, sodium bicarbonate, ammonia gas, ammonium hydroxide and sodium carbonate.

30. The process as claimed in claim 29, wherein the base is ammonium hydroxide.

31. The process as claimed in claim 26, wherein the anti-solvent is water.

32. The process as claimed in claim 26, wherein preparation of ivosidenib comprising the steps of: obtaining a solution of racemic ivosidenib of formula (XI) in a solvent;
(a) selectively crystallizing compound of formula (XII);
(b) separating the compound of formula (XII) by filtration and collecting the mother liquor;
(c) optionally epimerizing the compound of (XII) and repeating step (a) to (c); and
(d) isolating the Ivosidenib of Formula (I) from a mother liquor of step (c) or (d). 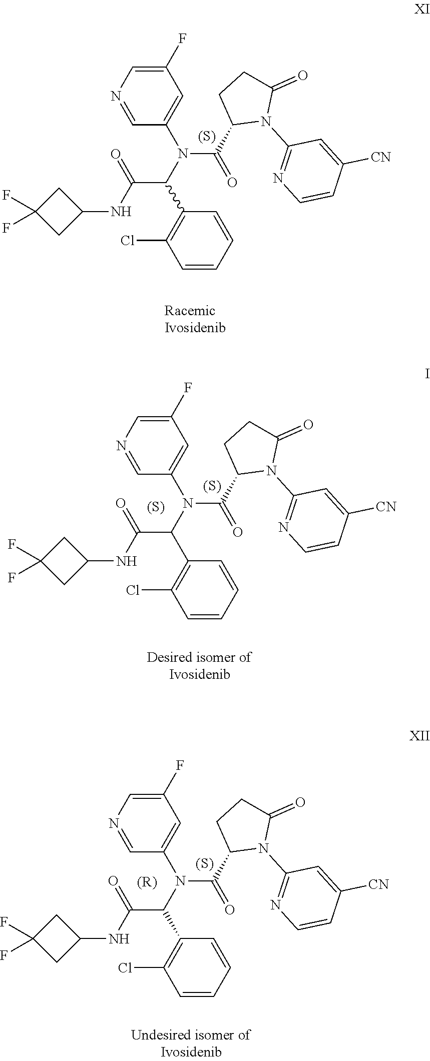
wherein racemic ivosidenib as represented formula (XI) is a racemic mixture of compound of Formula (XII) ((2S)—N-((1R)-1-(2-chlorophenyl)-2-((3, 3-difluoro cyclobutyl) amino)-2-oxoethyl)-1-(4-cyanopyridine-2yl)-N-(5-fluoropyddin-3-yl)-5-oxopyrrolidine-2-carboxamide) and compound Formula (I) ((2S)—N-((1S)-1-(2-chlorophenyl)-2-((3, 3-difluorocyclobutyl) amino)-2-oxoethyl)-1-(4-cyanopyridine-2yl)-N-(5-fluoropyridin-3-yl)-5-oxopyrrolidine-2-carboxamide).

33. The process as claimed in claim 32, wherein the solvent used in step (a) is selected from toluene, isopropanol, MTBE, anisole, chlorobenzene, ethyl acetate and mixture thereof.

34. The process as claimed in claim 33, wherein the suitable solvent is toluene.

35. The process as claimed in claim 32, wherein the epimerizing the compound of (XII) is carried out by treating the compound of (XII) with a suitable base, optionally in presence of suitable solvent.

36. The process as claimed in claim 35, wherein the suitable base used is selected from DBU, DABCO, DIPEA, N-methyl morpholine, piperazine, piperidine, cesium carbonate and tripotassium phosphate.

37. The process as claimed in claim 35, wherein the suitable solvent used is selected from THF, DCM, ethyl acetate, toluene, methanol, acetonitrile and acetone and mixture thereof.

38. A process of preparation of (3,3-difluorocyclobutyl) isonitrile according to the compound of formula (IV), comprising the step of: 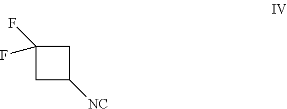
reacting (3,3-difluorocyclobutyl) formamide according to the compound of formula (II with phosgene derivative, optionally in presence of suitable solvent.

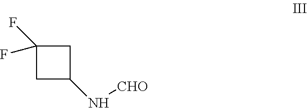

39. The process as claimed in claim 38, wherein the phosgene derivative is selected from phosgene, diphosgene and triphosgene.

40. The process as claimed in claim 38, wherein the solvent is selected from dichloromethane, chloroform, carbon tetrachloride, 1,2-dichloroethane, trichloroethylene THF, toluene, acetonitrile, tetrachloroethylene and mixture thereof.

41. The process as claimed in claim 38, wherein the reaction is carried out in the presence base selected from triethyl amine, diethyl amine, or diethyl isopropylamine.

42. The process as claimed in claim 41, wherein the base used is 2.5 to 3.0 mole equivalent with respect to 2-chloro benzaldehyde.

43. The process as claimed in claim 38, further comprising converting compound of formula (IV) to Ivosidenib of Formula (I).
